(19) United States
(12) Patent Application Publication
(10) Pub. No.: US 2016/0113484 A1
(43) Pub. Date: Apr. 28, 2016

(54) TROCAR, PORT, AND SURGERY ASSISTANCE SYSTEM

(71) Applicants: ADVANCED HEALTHCARE CO., LTD., Tokyo (JP); NATIONAL UNIVERSITY CORPORATION CHIBA UNIVERSITY, Chiba (JP); KYOCERA OPTEC CO., LTD., Tokyo (JP)

(72) Inventor: Toshiya NAKAGUCHI, Chiba-shi (JP)

(73) Assignees: ADVANCED HEALTHCARE CO., LTD., Itabashi-ku, Tokyo (JP); NATIONAL UNIVERSITY CORPORATION CHIBA UNIVERSITY, Chiba-shi, Chiba (JP); KYOCERA OPTEC CO., LTD., Ome-shi, Tokyo (JP)

(21) Appl. No.: 14/891,500

(22) PCT Filed: May 8, 2014
(86) PCT No.: PCT/JP14/62340

§ 371 Date: Nov. 16, 2015

(30) Foreign Application Priority Data
May 16, 2013 (JP) 2013-104527

(51) Int. Cl. A61B 1/313; A61B 17/34; A61B 1/018; A61B 1/12; A61B 1/00; A61B 17/02; A61B 1/05

(52) U.S. Cl. CPC A61B 1/3132; A61B 17/0218; A61B 17/3423; A61B 1/051; A61B 1/126; A61B 1/00009; A61B 1/00048; A61B 1/018; A61B 2090/366

(57) ABSTRACT

A technique using a trocar including a retractable camera in which fogging and organic matters are removed to clean a lens of the camera. The trocar includes a pipe portion and a head portion. The pipe portion includes a side opening portion. According to rotation of a changing over mechanism and a shaft, the retractable camera rotates such that the camera is changeable between a stored position and a deployed position by passing through the side opening portion. A wiper blade is fixed to an end face of the side opening portion on a tip side of the trocar. When the retractable camera rotates, a tip of the wiper blade contacts a camera lens in a deformable manner.

### TECHNICAL FIELD

The present invention relates to a trocar to be used in a laparoscopic surgery. However, the trocar is referred to as a port in a case where the trocar is used in a thoracoscopic surgery. The present invention further relates to a surgery assistance system utilizing the trocar as a core technology for surgery.

### BACKGROUND ART

In recent years, in order to maintain and enhance the QOL (quality of life) of patients, surgical operations of low invasiveness are being performed using laparoscopic surgery and so on. In abdominal laparoscopic surgery, carbonic acid gas is injected into the abdominal cavity so that the abdominal wall is distended, and thereby space and a good field of view for manipulation are ensured. A small hole is formed in the abdominal wall, and an instrument called a trocar is inserted through the hole. Then, usually, a laparoscope (i.e. a CCD camera) and a forceps (which is a surgical instrument) are inserted into the interior of the body of the patient, and the required surgical operation is performed while observing an image displayed upon a monitor by the laparoscope.

However, in some cases, fogging of a lens of laparoscope and/or adhesion of organic matters (e.g., oil film and tissue fragment, more specifically, blood, flesh, and body fluid) to the lens of laparoscope may make it impossible to secure a surgical field during laparoscopic surgery. To solve the problem, the laparoscope is withdrawn from the trocar every time it is required, and the lens of the laparoscope is cleaned up to be subsequently returned into the trocar. As a result, the surgical operation is temporarily interrupted every time the lens requires cleaning-up.

To solve the above described problem, various fogging preventing measures, fogging eliminating measures, and organic matter removing measures are studied. For example, patent literature 1 proposes a technology of heating a distal end of a laparoscope to keep the laparoscope at a temperature identical to that of the patient body. This technology makes it possible to prevent a lens of the laparoscope from being fogged due to a difference in temperature between a temperature of an operating room and the temperature of the patient body.

### CITATION LIST

### Patent Literature

### [PATENT LITERATURE 1]

### JP 2001-299678A

### SUMMARY OF INVENTION

### Technical Problem

The technology can prevent a lens of the laparoscope from being fogged due to the difference in temperature. However, the technology cannot serve to remove organic matters. Further, the technology requires the laparoscope to have an additional heating unit for removing the fogging. This makes a structure of the laparoscope complicated. That is, the technology of the patent literature 1 is impractical. Under the situation, currently, there is no drastic measure against fogging and adherence of organic matters. Therefore, to solve the above problem, the following operation is repeated: The laparoscope is withdrawn from a trocar, a lens of the laparoscope is cleaned up, and then the laparoscope is inserted into the trocar again. The lens cleaning operation is usually repeated more than 10 times during a single surgical procedure. The lens cleaning operation is not involved in the basic surgical procedure, which means that these operations put a burden upon a surgeon.

As described above, the surgeon performs laparoscopic surgery while observing an image obtained by a laparoscope. Accordingly, if the laparoscope is once withdrawn from a trocar and subsequently returned into the trocar, the image before withdrawing the laparoscope may differ from an image after the laparoscope is returned. Therefore, the surgeon needs to confirm an object for operation (e.g., an organ) again. This also puts another burden upon the surgeon.

Now, the inventor of the present invention proposes a trocar having a retractable camera (detailed description follows). The camera is changed over between a stored position and a deployed position within an abdominal cavity of a patient. Meanwhile, the trocar having a retractable camera is a core technology that realizes a virtual abdominal surgery in which a three-dimensional real-time image is used.

On the contrary, a trocar having a retractable camera has problems as listed below.

More specifically, once the trocar is inserted into an interior of a body of a patient through an abdominal wall of a patient, the trocar will not be withdrawn until the surgical procedure ends. Therefore, it is impossible to remove fogging and organic matters by taking the camera out of an abdominal cavity of the patient, as in the above described conventional case that the laparoscope is withdrawn to remove fogging and organic matters.

A purpose of the present invention is to solve the above described problem. More specifically, the purpose of the present invention is to clean up a lens of a camera of a trocar having a retractable camera by removing fogging and organic matters of the camera.

Further purpose of the present invention is to reduce a burden put upon a surgeon in comparison with a lens cleaning operation required in the conventional typical laparoscope.

Still further purpose of the present invention is to prevent an image from being misaligned during the lens cleaning operation and, thereby, to reduce a burden put upon a surgeon.

### Solution to Problem

The present invention that solves the above problem is directed to a trocar having a pipe portion that inserts a surgical instrument into an interior of a body of a patient, the trocar including a side opening portion that is provided at a position of the pipe portion that, during surgery, is within the body of the patient, a retractable camera that is rotatably disposed so that it can be changed over between a stored position in which it is stored within the pipe portion and a deployed position in which it is deployed via the side opening portion to an exterior of the pipe portion and is capable of obtaining an image, and a lens cleaning unit that contacts a camera lens for cleaning while the retractable camera is rotated.

In the present invention, further preferably, the lens cleaning unit includes a base portion and a tip portion, the base portion being fixed to a tip side of the side opening portion, the tip portion being deformable when contacting the camera lens.

This ensures lens cleaning operation, i.e., removal of fogging and organic matters of a camera, in a trocar equipped with a retractable camera.

The lens cleaning operation of the conventional typical laparoscope necessitates removal, cleaning, and insertion of a laparoscope, whereas the laparoscope of the present invention involves just a simple lens cleaning operation that can reduce a burden put upon a surgeon.

Further preferably, the present invention includes an energizing unit that energizes the retractable camera toward a deployed position.

The energizing unit ensures that the retractable camera returns to the same position (deployed position) after cleaning operation. As a result thereof, the present invention can prevent misalignment of an image caused by the lens cleaning operation and thereby reduce a burden put upon a surgeon.

The present invention that solves the above described problem is directed to a surgery assistance system including the above described trocar, the laparoscope, and an image processing device that performs processing to combine an image obtained from the laparoscope and an image obtained from the retractable camera, wherein the image processing device includes a partial interruption instruction unit that provides an instruction, when the retractable camera is changed over from the deployed position to the stored position, to use an image lastly obtained at the deployed position.

The present invention that solves the above described problem is directed to a surgery assistance system including a plurality of the above described trocars, and an image processing device that performs processing to combine an image obtained from the laparoscope and an image obtained from the retractable camera, wherein the image processing device includes a partial interruption instruction unit that provides an instruction, when the retractable camera is changed over from the deployed position to the stored position, to use an image lastly obtained at the deployed position.

In the present invention, further preferably, the image processing device includes an image matching determination unit that determines whether or not the last image matches newly obtained latest image when the retractable camera is returned from the stored position to the deployed position, and a restart instruction unit that provides an instruction to use the latest image when the image matching determination unit confirms matching of images.

Various functions provided by the partial interruption instruction unit, the image matching determination unit, the restart instruction unit, etc, can eliminate inconveniences that occur in an image processing device resulting from the lens cleaning operation.

Further preferably, the present invention further includes a projector that is provided above an operating table, and that projects the combined image onto a position corresponding to an abdomen of the patient.

The present invention that solves the above described problem is directed to a port having a pipe portion that inserts a surgical instrument into a lung of a patient and being provided in a chest wall via the pipe portion, wherein the port includes a side opening portion provided in a position of the pipe portion that is placed within the lung of the patient, a retractable camera that is rotatably disposed so that it can be changed over between a stored position in which it is stored within the pipe portion and a deployed position in which it is deployed via the side opening portion to the exterior of the pipe portion and capable of obtaining an image, and a cleaning unit that contacts a camera lens for cleaning while the retractable camera is rotated.

### Advantageous Effect of Invention

According to the present invention, a trocar with a retractable camera ensures a lens cleaning operation in which fogging and organic matters of a camera are removed.

Further, the present invention can reduce a burden put upon a surgeon, as compared to the lens cleaning operation performed in the conventional typical laparoscope.

Still further, the present invention can prevent misalignment of an image caused by the lens cleaning operation and thereby reduce a burden put upon a surgeon.

### BRIEF DESCRIPTION OF DRAWINGS

FIG. 1 illustrates a trocar equipped with a retractable camera.

FIG. 2 illustrates modifications of a lens cleaning unit (including a wiper blade).

FIG. 3 illustrates a modification of the trocar equipped with a retractable camera.

FIG. 4 illustrates a surgery assistance system.

FIG. 5 is a functional block diagram of an image processing device.

FIG. 6 illustrates a basic principles of a depth estimation.

FIG. 7 illustrates a modification of the surgery assistance system.

FIG. 8 illustrates another modification of the surgery assistance system.

### DESCRIPTION OF EMBODIMENTS

<Trocar Equipped with Retractable Camera>
˜Structure˜

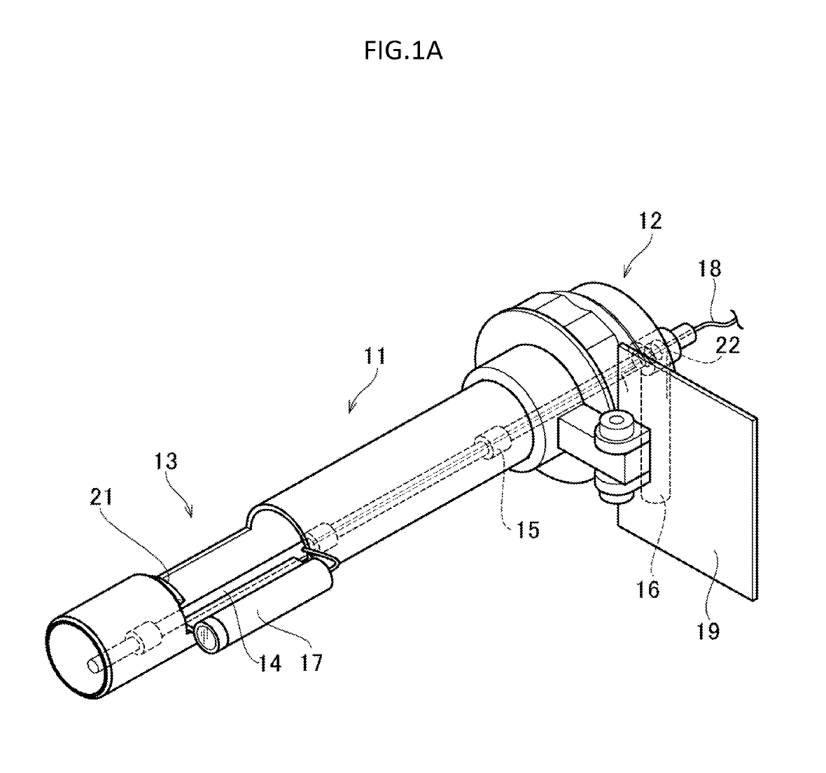

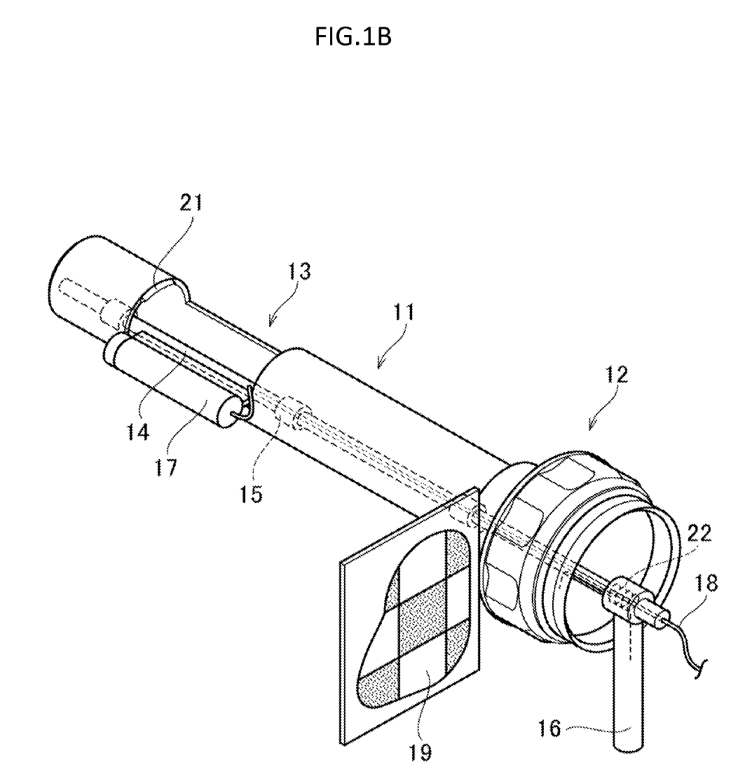

A structure of a trocar having a retractable camera 17 will be described below. FIG. 1 includes perspective views of a trocar equipped with a retractable camera. FIG. 1(a) and FIG. 1(b) are illustrated viewed from different directions.

A trocar 1 includes a pipe portion 11 and a head portion 12. Most of the pipe portion 11 is inserted into an interior of a hole provided in an abdominal wall of a patient. The head portion 12 is continuous to an upper portion of the pipe portion 11. The head portion 12 includes a hollow portion therein so that a forceps can be inserted through the upper portion of the head portion 12. Further, though a detailed description is omitted here, the head portion 12 includes a sealing mechanism and an air blowing mechanism. The sealing mechanism prevents air from leaking at the time of insertion/withdrawal of the forceps. The air blowing mechanism blows air into an interior of an abdominal cavity of the patient.

A side opening portion 13 is provided at a position in the pipe portion 11 that is definitely placed in an interior of a body of a patient. A shaft 14 is disposed such that it is oriented in an axis direction of the pipe portion and it is disposed along one edge of the side opening portion 13. A plurality of bearings 15 are fixed to an inner wall of the pipe portion 11, and these bearings 15 hold the shaft 14 such that the shaft 14 can rotate (pivot) within the bearings 15. An end portion of the shaft 14 projects to the exterior of the trocar. A changing over mechanism 16 is provided on the end portion of the shaft 14. This changing over mechanism 16 is configured to be changeable between a stored position and a deployed position.

The retractable camera 17 is rigidly and integrally coupled to the shaft 14 at a position corresponding to the side opening portion 13. Accordingly, the retractable camera 17 rotates, according to rotation of the changing over mechanism 16 and the shaft 14, changeably between a stored position in which it is stored within the pipe portion and a deployed position in which it is deployed exterior of the pipe portion via the side opening portion 13 and capable of obtaining an image. A cable 18 is connected to the retractable camera 17, and the cable 18 is led out through the trocar 1 and is connected to an external image processing device 6.

It should be understood that while, in the shown configuration, the cable 18 is led out along the shaft 14, it would be even more desirable to make the shaft 14 hollow, and to lead out the cable 18 through the shaft 14; in this case, there would be no danger of the cable 18 becoming disconnected during insertion of the forceps.

The mark 19 is provided upon the head portion 12 of the trocar. In this embodiment, a white and black checkered flag pattern is shown as one example of this mark 19, but this is not to be considered as being limitative; any mark that can be recognized by the optical tracking sensor 9 will be acceptable.

Characterized structure of the present invention will be described below. A wiper blade 21 is fixed to an end face of the side opening portion 13 on a tip side of the trocar.

The wiper blade 21 has a cross sectional shape that is sharp like a blade in its tip end. The wiper blade 21 is made of rubber. These characteristics renders suitable rigidity to a base of the wiper blade 21 and suitable flexibility to a tip of the wiper blade 21. The side opening portion 13, the retractable camera 17, and the wiper blade 21 have a positional relationship as set forth below. That is, rotation of the retractable camera 17 ensures a contact between the tip of the wiper blade 21 and a camera lens such that the tip of the wiper blade can be slightly deformed on the camera lens.

Meanwhile, in a case of a vehicle, a wiper blade rotates in such a manner that the wiper blade slides over a front glass of the vehicle. To the contrary, in a case of the wiper blade 21, the wiper blade 21 is fixed, and the retractable camera 17 rotates to cause the wiper blade 21 and a camera lens to contact each other. In other words, the wiper blade 21 is configured based on an inverted concept rather than a concept of a wiper blade of a vehicle.

The changing over mechanism 16 is provided with a torsion spring 22. The torsion spring 22 may be trapped in an inner space of the changing over mechanism 16. One end of the torsion spring 22 is fixed to an interior wall of the head portion 12, and the other end of the torsion spring 22 is fixed to the changing over mechanism 16. Normally, an elastic force of the torsion spring 22 energizes the retractable camera 17 such that the retractable camera 17 is deployed by the elastic force of the torsion spring 22 via the shaft 14. In other words, both of the changing over mechanism 16 and the retractable camera 17 keep the respective deployed positions. When the changing over mechanism 16 is activated to rotate, the retractable camera 17 resists against the elastic force of the torsion spring 22 and passes through the side opening portion 13 to be stored.

˜Operation˜

A changing over operation of the retractable camera 17 will be described below. A surgeon operates the changing over mechanism 16.

In a state that the changing over mechanism 16 is not activated, the retractable camera 17 keeps its deployed position with the help of energization of the torsion spring 22. While the pipe portion 11 is inserted into a hole in an abdominal wall of a patient, the changing over mechanism 16 is activated to be at the stored position and is caused to keep the stored position. The retractable camera 17 is thus placed at its stored position via the shaft 14. This positioning of the retractable camera 17 facilitates insertion of the pipe portion 11 into the hole in the abdominal wall of the patient without being hindered by the retractable camera 17 (trocar insertion: deployed position→stored position).

After the insertion of the pipe portion 11, when the changing over mechanism 16 is released, the retractable camera 17 is changed over toward the deployed position via the shaft 14. In this state, the retractable camera 17 performs an image capturing operation (image capturing: stored position→deployed position).

During laparoscopic surgery, space and a good field of view for manipulation sometimes cannot be ensured due to fogging of a lens of the retractable camera 17 and attachment of organic matters (e.g., oil film and tissue fragment, more specifically, blood, flesh, and body fluid) to the lens of the retractable camera 17.

In such case, the changing over mechanism 16 is once activated and subsequently the changing over mechanism 16 is released. This causes the retractable camera 17 to be changed over from its deployed position to its stored position and subsequently changed over from its stored position to its deployed position. According to this reciprocating rotation, a surface of the camera lens contacts and slides over the wiper blade 21. A tip of the wiper blade 21 is slightly deformed, and the deformation generates a pressure force that effects on the surface of the camera lens. As a result thereof, fogging and organic matters of the camera lens are removed to complete the lens cleaning operation (lens cleaning: deployed position→stored position→deployed position). If a satisfactory result cannot be obtained during the lens cleaning operation, the changing over operation is repeated once more.

After the lens cleaning operation, the retractable camera 17 is surely returned to the position (deployed position) where the retractable camera 17 was before the lens cleaning operation with the help of the energization of the torsion spring 22. This allows the retractable camera 17 to restart image capturing operation.

After surgical procedure, when the pipe portion 11 is withdrawn, the changing over mechanism 16 is activated to be at its stored position and to keep the stored position. Accordingly, the retractable camera 17 is placed at its stored position via the shaft 14. This allows the pipe portion 11 to be withdrawn through the abdominal wall of the patient without the withdrawing operation being hindered by the retractable camera 17 (withdrawal of trocar: deployed position→stored position).

˜Effects˜

(1) As described in the operations of the present embodiment, a trocar with a retractable camera can remove fogging and organic matters of a camera and clean up a lens of the camera.

(2) The lens cleaning operation of the present embodiment involves only activation and release of the changing over mechanism 16. More specifically, it is required for the surgeon to only operate the changing over mechanism 16 from a deployed position to a stored position. Energization of the torsion spring 22 causes the changing over mechanism 16 to return to the deployed position from the stored position.

To the contrary, in the lens cleaning operation required in the conventional typical laparoscope, withdrawal and insertion of the laparoscope are essential. In view of the above, the present embodiment can reduce a burden put upon a surgeon, comparing to the lens cleaning operation of the conventional typical laparoscope.

(3) Further, in the present embodiment, the retractable camera 17 resides in the same position (deployed position) both before and after the lens cleaning operation. Meanwhile, a trocar is secured by a shrinkage pressure of an abdominal wall of a patient. This ensures prevention of possible misalignment of an image caused during the lens cleaning operation. This contributes to reduction of a burden put upon a surgeon.

Further, the reduction of possible misalignment of an image can decrease a burden of arithmetic processing of the image processing device 6 (will be described below in detail).

(4) The present embodiment has such a simple structure that a trocar with a retractable camera is provided with the wiper blade 21. This simple structure ensures an easy manufacturing, almost no possibility of malfunction, good durability, and high safety.

### ˜Modification of Lens Cleaning Unit˜

The wiper blade 21 is exemplified as, but not limited to, the lens cleaning unit that removes fogging and organic matters to clean up a lens. In order for a surface of a camera lens to contact and slide over the lens cleaning unit, the lens cleaning unit includes a tip portion having a suitable softness and a base portion having a suitable rigidity. The rigidity of the base portion ensures resilience to the sliding-over pressure. With this characteristics, the base portion of the lens cleaning unit is fixed to an end face of the side opening portion 13 on a tip side of the side opening portion 13, and a tip portion of the lens cleaning unit becomes deformable by being brought into contact with the lens. Because of the softness and the deformability of the tip, a surface of the lens is not damaged. Any shape and any material that can realize such characteristics are acceptable.

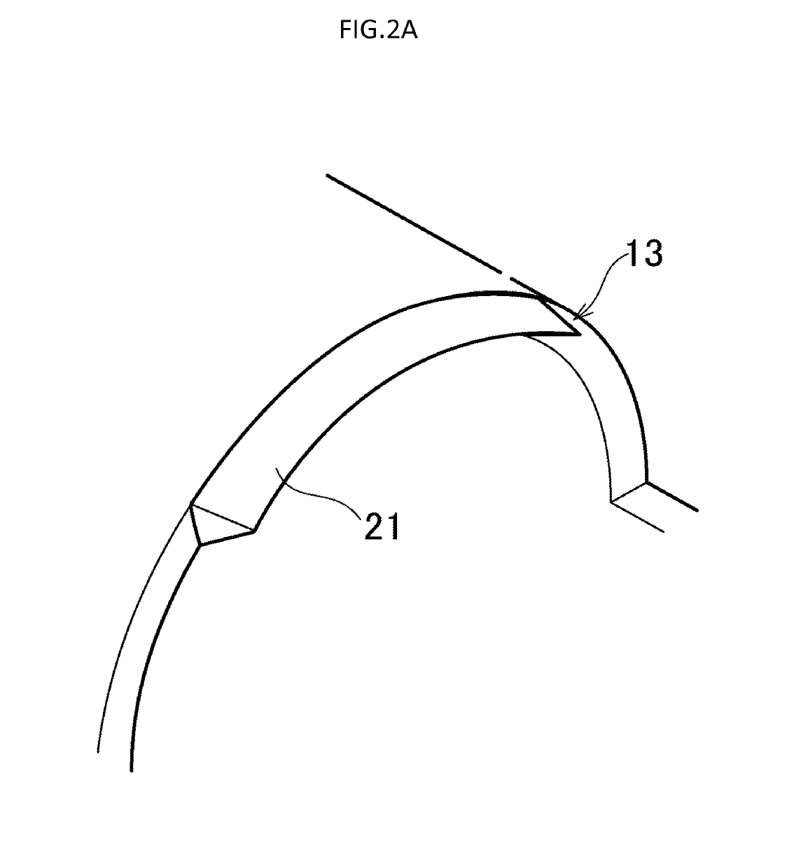

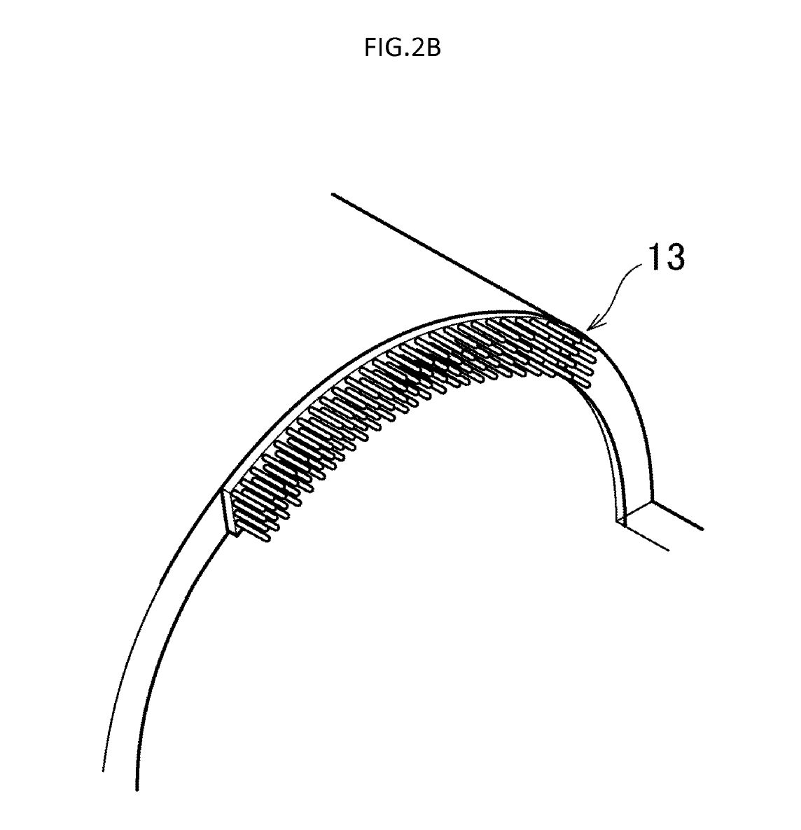

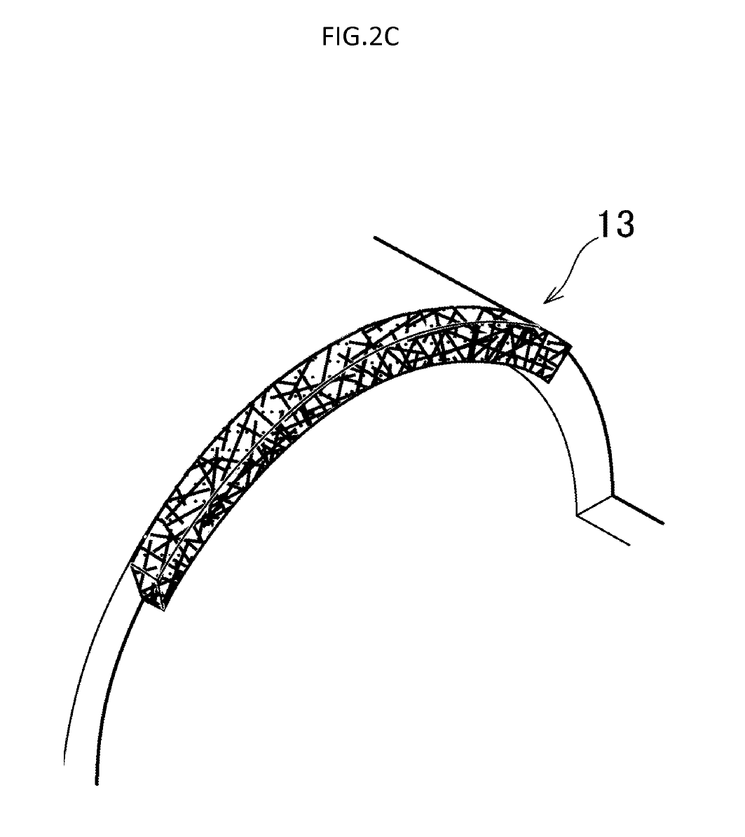

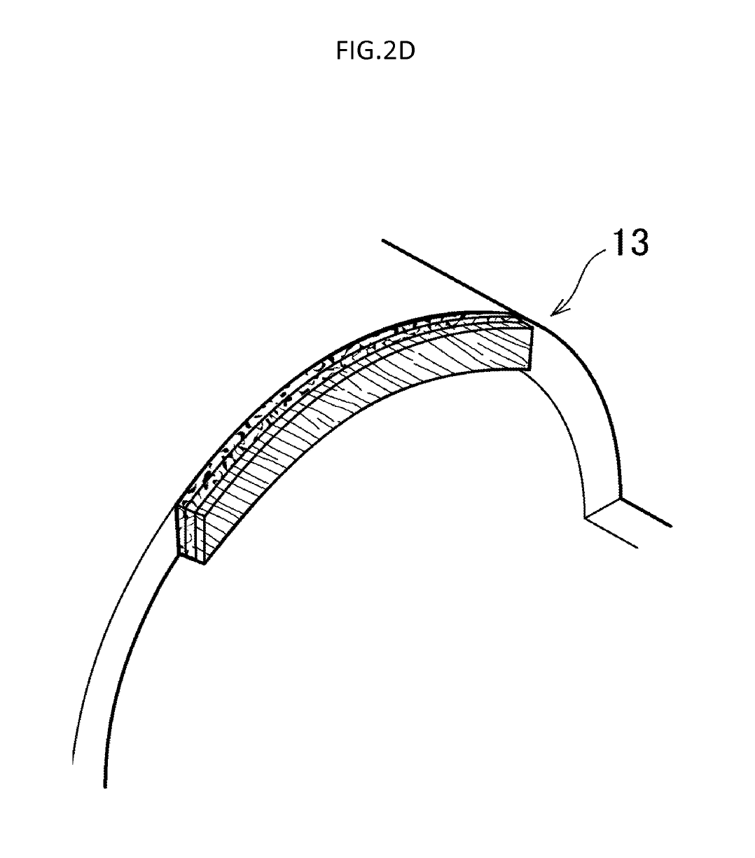

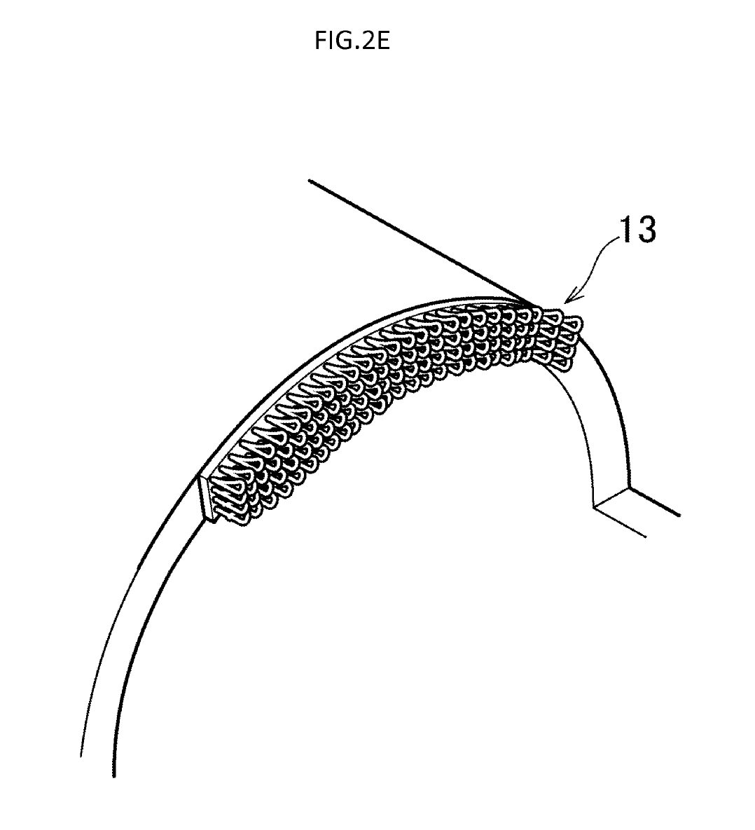

FIG. 2 illustrates examples of a lens cleaning unit including the wiper blade 21. FIG. 2(a) is the wiper blade 21. The wiper blade 21 may be made of a resin instead of rubber.

FIG. 2(b) is a brush. Many brush bristles are implanted on the end face of the side opening portion 13 on the tip side of the side opening portion 13. The brush bristles have a suitable rigidity and softness. The brush bristles may be made of resin such as rubber or may be made of natural fibers. A length of the brush bristles can be adjusted and may be formed into a blade shape.

FIG. 2(c) illustrates an example of the wiper blade made of fibers that are entangled irregularly, e.g., a loofah sponge, and formed into a flat plate shape. The fibers may be dense on a base side and may be rough on a tip side. FIG. 2(d) illustrates an example of the wiper blade made of clothes that are laminated together and formed into a flat plate shape. The laminated clothes on the base side may have high rigidity and the laminated clothes on the tip side may have low rigidity. FIG. 2(e) illustrates an example of the wiper blade made of a fabric (e.g., towel) having pile texture and formed into a flat plate shape. The fibers may be made of any material.

### ˜Modification of Retractable Camera Mechanism˜

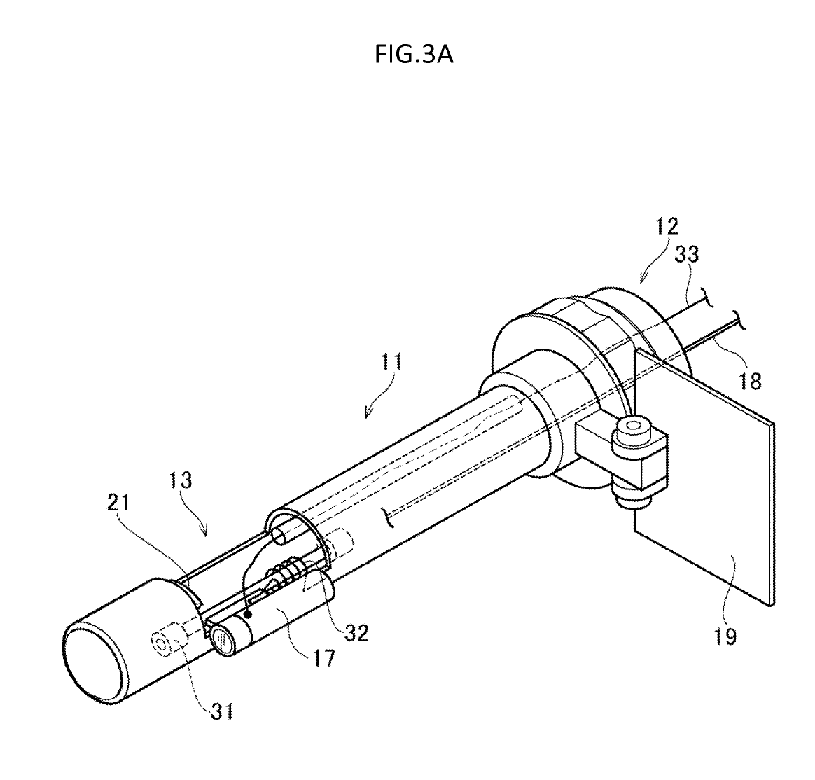

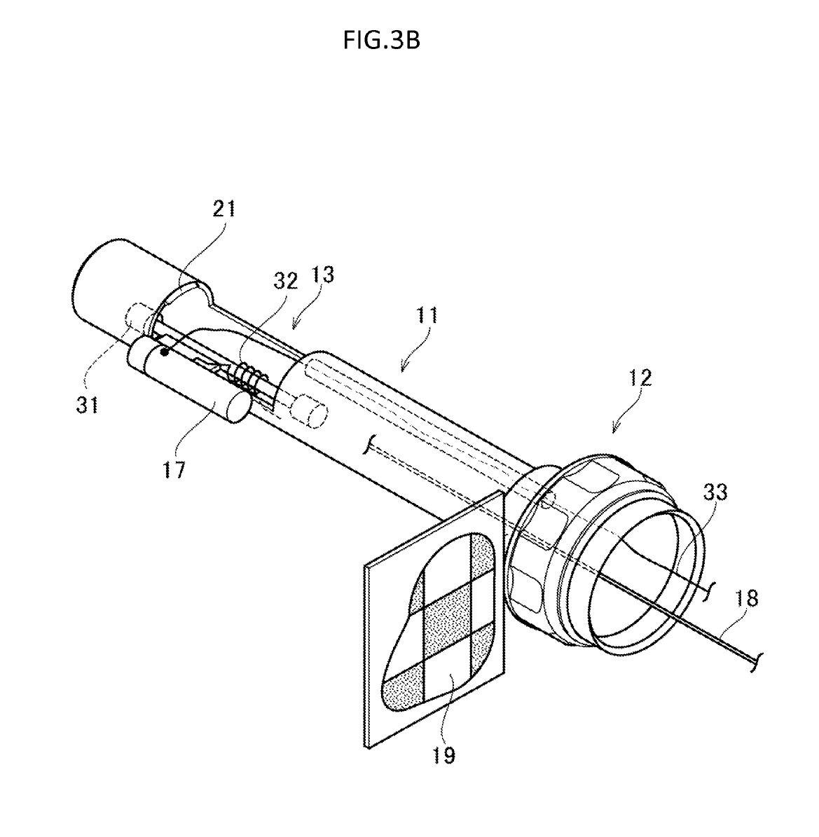

The retractable camera mechanism is not limited to the above described structure. FIG. 3 is a perspective view of a trocar 2 as a modification. FIG. 3(a) illustrates the retractable camera 17 that is deployed to its deployed position, and FIG. 3(b) illustrates the retractable camera 17 that is stored in its stored position. Elements common to those of FIG. 1 are assigned with the same numbers or symbols. The trocar 2 includes the pipe portion 11 and the head portion 12. The side opening portion 13 is provided at a position of the pipe portion 11 that is placed in an interior of a body of a patient.

A rotatable hinge mechanism 31 is provided on one edge of the opening portion that is oriented to an axis direction of the pipe portion. The retractable camera 17 is coupled to the pipe portion 11 via the hinge mechanism 31. The hinge mechanism 31 is provided with a torsion spring 32. Normally, the torsion spring 32 has an elastic force that energizes the retractable camera 17 such that the retractable camera 17 is deployed.

A tension cable 33 that protrudes to the exterior of the trocar is coupled to the retractable camera 17. When the tension cable 33 is pulled, the retractable camera 17 resists against the elastic force of the torsion spring 32 and is stored passing through the side opening portion 13. A cable 18 is connected to the retractable camera 17. A mark 19 is provided on the head portion 12.

Meanwhile, the tension cable 33 is protected by a guide so that a danger of disconnection of the tension cable 33 that may occur when the forceps 4 is inserted or pulled out is decreased.

When the pipe portion 11 is inserted into an interior of a hole in an abdominal wall of a patient, the tension cable 33 is pulled to keep the retractable camera 17 at its stored position. After the insertion of the pipe portion 11, the tension cable 33 is released and thereby the camera 17 is placed at the deployed position. An image is captured in this state. When the pipe portion 11 is withdrawn after the surgical procedure ends, the tension cable 33 is pulled and thus the retractable camera 17 is returned to its stored position.

For cleaning a lens, the tension cable 33 is pulled and subsequently the tension cable 33 is released. With such operation, the retractable camera 17 is changed over from its deployed position to its stored position, and is subsequently changed over from its stored position to its deployed position.

Operation and effects of the wiper blade 21 is identical to what is described above. The torsion spring 32 has a structure similar to the torsion spring 22, and thus is operated similarly and produces similar effects.

Meanwhile, the torsion spring 22 and the torsion spring 32 are exemplified as the energizing unit but a flat spring, etc., may also be employed as the energizing unit.

### <Surgery Assistance System>

˜Summary˜

The present inventor proposes a surgery assistance system that realizes, based on a trocar with a retractable camera as a core technology, a virtual abdominal surgery in which a three-dimensional real-time image is used (detailed description will follow).

Meanwhile, during the lens cleaning operation, it becomes impossible for the retractable camera 17 to capture an image of an object for operation (e.g., organ) for a while, because the retractable camera 17 is placed at a stored position and temporarily stored at the stored position. As a result thereof, there may occur various inconveniences.

In the present embodiment, a characteristic control eliminates these inconveniences.

˜Structure˜

A surgery assistance system 101 in which a three-dimensional real-time image is used will be described below. FIG. 4 is a schematic diagram of the surgery assistance system 101.

The surgery assistance system 101 includes forceps trocars 1a and 1b, a laparoscope trocar 3, forcipes 4a and 4b, a laparoscope 5, an image processing device 6, a three dimensional monitor 7, and an optical tracking sensor 9. The forceps trocars 1a and 1b include retractable cameras 17a and 17b and marks 19a and 19b, respectively. The laparoscope 5 includes a mark 19d. The image processing device 6 inputs images obtained from the retractable cameras 17a and 17b and an image obtained from the laparoscope 5 and combines these images to create a three dimensional image. The three dimensional monitor 7 outputs the three dimensional image created by the image processing device 6.

The forcipes 4a and 4b are one type of surgical instrument, and are used for grasping, holding down, pulling, and cutting blood vessels and organs and so on. Each of them is generally formed as a pair of scissors, and its inner end portion is operated by outer gripping portions being rotated around a fulcrum. When the gripping portions are closed together, these forcipes can be inserted through the forceps trocar 1a and 1b. It should be understood that while, generally, a plurality of forcipes are used in abdominal laparoscopic surgery, at least one forceps and one forceps trocar are enough for application of this system.

The laparoscope 5 is one type of endoscopic instrument, and includes a camera. The laparoscope 5 is inserted into an interior of a body of a patient by being passed through the laparoscope trocar 3. The mark 19d is provided at a position on the laparoscope 5 that is not placed in the interior of the body of the patient.

The optical tracking sensor 9 measures a three-dimensional position of the respective marks 19a, 19b, and 19d and outputs a measurement result to the image processing device 6. Meanwhile, in the present embodiment, the optical tracking sensor 9 recognizes white portions and black portions on the marks as visible rays. The optical tracking sensor 9 may emit infrared light and receive infrared light reflected by the marks. The optical tracking sensor 9 is not limited to an optical tracking sensor but may be a magnetic sensor that can at least measure a three-dimensional position.

### ˜Effect of Entire System˜

The laparoscopic surgery being applied with the surgery assistance system 101 is performed based on the normal laparoscopic surgery, more specifically, is not largely different in surgical form from that of the normal laparoscopic surgery. This allows the surgeon to use the knowledge and experiences the surgeon stored and accumulated to date as to the surgical procedure.

Further, the surgery assistance system 101 has a simple structure in which a trocar with a retractable camera is used. Thus, the existing surgery assistance system can be used only by making a simple enhancement to the system.

Meanwhile, in the widely performed conventional laparoscopic surgery, only a narrow surgical field could be obtained since the laparoscopic surgery was performed relying upon only an image obtained from a laparoscope. Specifically, image information as to a depth could not be obtained. If a new hole is provided in an abdominal wall of a patient for the purpose of inserting another camera that performs a three-dimensional shape measurement of good accuracy, the low invasive property is degraded.

In the present embodiment, with the use of trocars 1a and 1b having retractable cameras 17a and 17b, respectively, a plurality of cameras can be inserted into the abdominal cavity of the patient. At the time, it is not necessary to make a new hole in the abdominal wall for the purpose of using a forceps trocar. Accordingly, the three-dimensional shape can be measured as well as the low invasive property can be maintained.

Further, the image processing device 6 creates a three-dimensional image and outputs a three-dimensional real-time image to the three-dimensional monitor 7. A surgeon can obtain a wide surgical field including depth information when he observes the three-dimensional monitor 7. This can reduce a burden put upon the surgeon.

˜Control˜

A basic control of the image processing device 6 will be described below. FIG. 5 is a functional block diagram of the image processing device 6. For convenience of description, a structure of the image processing device 6 is simplified.

The image processing device 6 includes an image inputting unit 61, an objective point extraction unit 62, a mark position input unit 63, a camera position estimation unit 64, a depth estimation unit 65, an image combining unit 66, and an image outputting unit 67.

The image inputting unit 61 inputs an image from each of the retractable cameras 17a and 17b and the laparoscope 5 via the respective cables 18.

The objective point extraction unit 62 searches the image (image obtained from each of the cameras 5, 17a and 17b) to extract an objective point for the image. For example, the objective point is extracted per pixel unit. Then, correspondency of the objective point between images is confirmed.

The mark position input unit 63 inputs three-dimensional positions of the marks 19a, 19b, and 19d via the optical tracking sensor 9. The mark 19 is secured to the trocar 1. At the time, the retractable camera 17 keeps its deployed position. In other words, a positional relationship between the mark 19 and the retractable camera 17 is invariant. The camera position estimation unit 64 can estimate three-dimensional positions of the cameras 17a and 17b based on the three-dimensional positions of the marks 19a and 19b. Similarly, based on the three-dimensional position of the mark 19d, a three-dimensional position of a camera of the laparoscope 5 can be estimated. This enables estimation of a distance L between cameras.

Meanwhile, it is also possible to cause the optical tracking sensor 9 to estimate camera positions, and the image processing device 6 may input thus estimated camera positions.

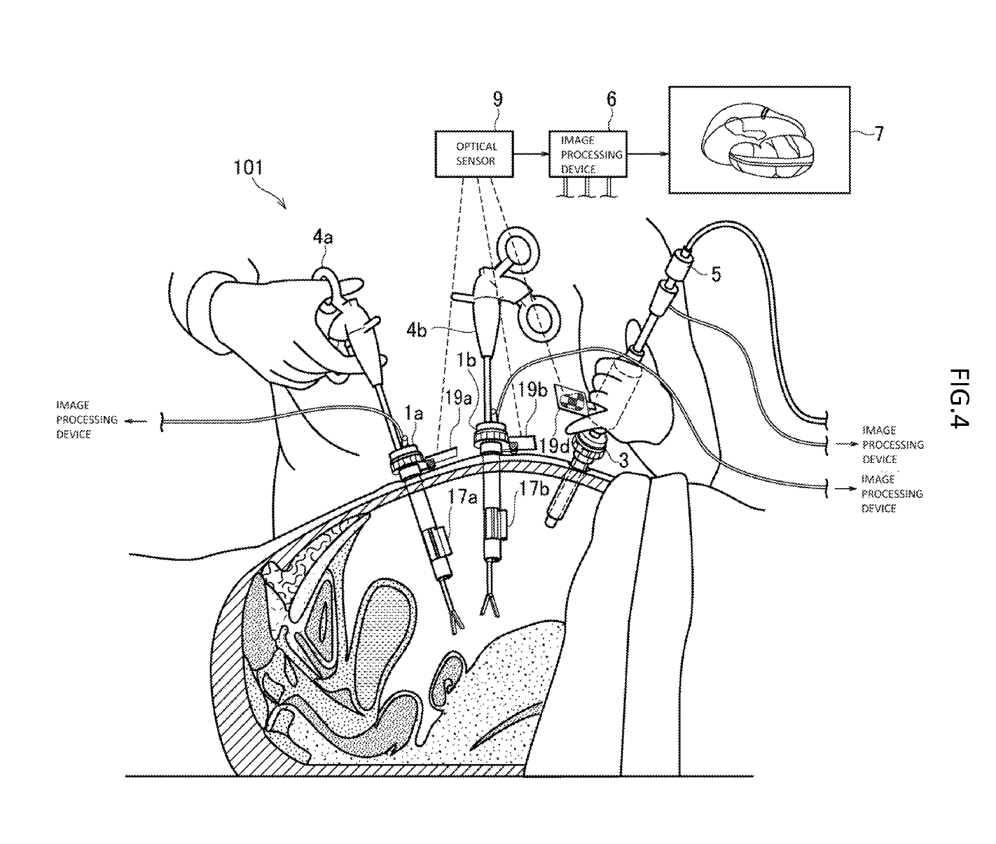

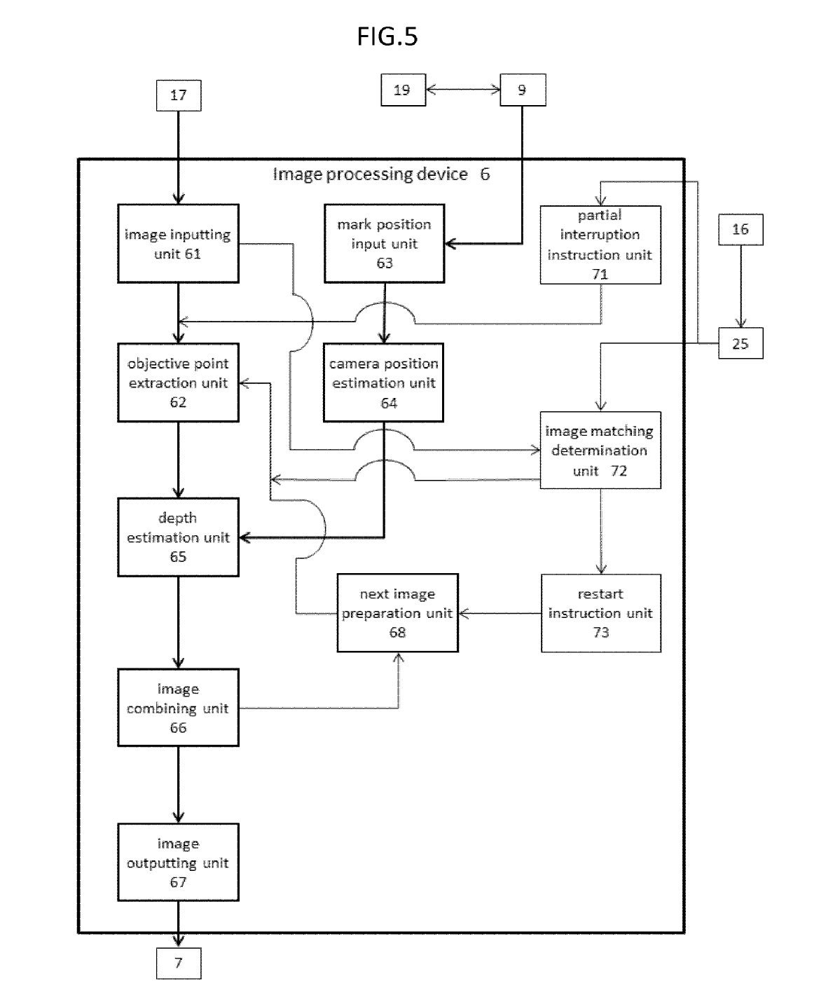

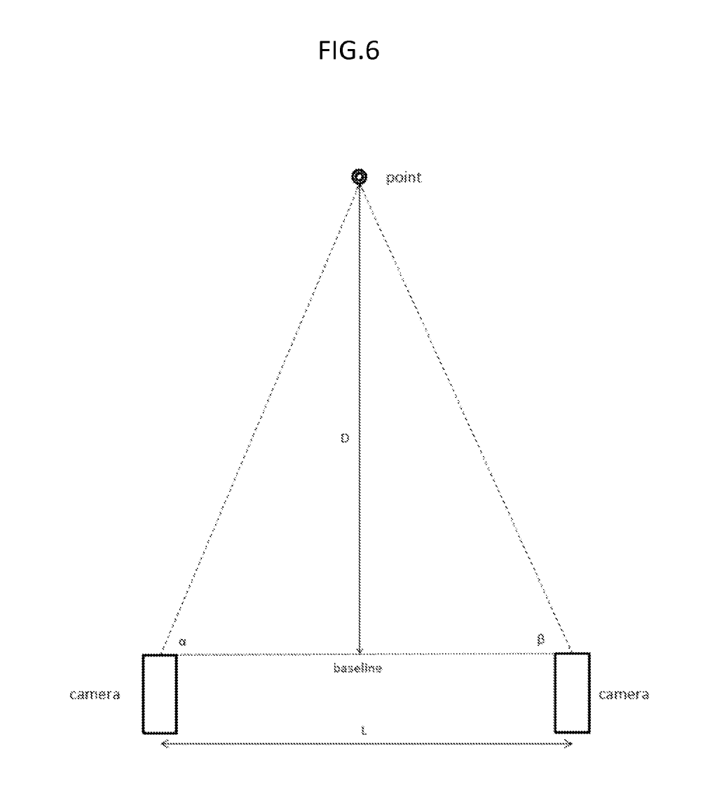

The depth estimation unit 65 estimates a depth D based on a triangle formed by two cameras and an objective point. FIG. 6 is a schematic diagram illustrating a basic principle of three-dimensional shape measurement. The depth D can be estimated, in a triangle formed by two cameras and an objective point, based on the distance L between two cameras, an angle α formed between a camera-camera base line and one camera's eye, and an angle 13 formed between a camera-camera base line and the other camera's eye. Meanwhile, increase of the number of cameras creates more number of triangles. This improves estimation accuracy.

The objective point extraction unit 62 and the depth estimation unit 65 repeat the above described control while moving the objective point, thereby measuring a three-dimensional shape of the object for operation. The image combining unit 66 creates a three-dimensional image by combining images based on the three-dimensional shape measurement result.

The image outputting unit 67 outputs a three-dimensional image to the three-dimensional monitor 7.

Further, the image processing device 6 includes a next image preparation unit 68. Based on the camera images and positional information that are input in real time, three-dimensional images are repeatedly output and thereby real-time images are provided. In other words, one-input-to-one-output control is repeated. At the time, if the control is repeated without making adjustment for each routine work, a burden of the image processing device 6 suffered from arithmetic processing will increase. Frequently, there is no large change between a currently creating image and a previously created image. Therefore, the next image preparation unit 68 temporarily stores information obtained in the control per routine work. The objective point extraction unit 62 and the depth estimation unit 65 reduce the burden suffered from the arithmetic processing by using information of the previous image (several images including a just-before image). More specifically, a difference from the previous image is utilized.

A characterized control of the present embodiment will be described below. The image processing device 6 includes a partial interruption instruction unit 71, an image matching determination unit 72, and a restart instruction unit 73. The changing over mechanism 16 of the trocar 1 is provided with a changed-over position detection sensor 25. The changed-over position detection sensor 25 detects a stored position/deployed position of the changing over mechanism 16.

The partial interruption instruction unit 71 causes the changed-over position detection sensor 25 to input a detection signal and, when it determines that the changing over mechanism 16 is changed over from a deployed position to a stored position, the partial interruption instruction unit 71 instructs the objective point extraction unit 62 to use not a newly obtaining latest image but the last image obtained at the deployed position (several images including an image just before changed-over).

The objective point extraction unit 62 stops searching the image that was instructed by the partial interruption instruction unit 71 and utilizes the last image. The objective point extraction unit 62 utilizes the latest image when no instruction about image was received from the partial interruption instruction unit 71.

The image matching determination unit 72 causes the changed-over position detection sensor 25 to input a detection signal and, when it determines that the changing over mechanism 16 was changed over from a stored position to a deployed position, the image matching determination unit 72 confirms matching between the last image and the newly obtained latest image (an image just after changed-over). If a difference between the images falls within a predetermined range, the image matching determination unit 72 determines that the images match each other.

The restart instruction unit 73 inputs a determination result of the image matching determination unit 72 to the effect that the images match each other and outputs a restart instruction to the next image preparation unit 68. The next image preparation unit 68 uses information of the last image. The objective point extraction unit 62 and the depth estimation unit 65 partially omit the arithmetic processing.

However, when the image matching determination unit 72 determines that the images do not match, the objective point extraction unit 62 and the depth estimation unit 65 start the arithmetic processing from the beginning without going through the next image preparation unit 68.

### ˜Effects of Characteristic Control˜

Various inconveniences may occur in the surgery assistance system during a lens cleaning operation. These inconveniences can be made better by a characteristic control.

(1) When the retractable camera 17 is placed in its stored position, it becomes impossible for the retractable camera 17 to continue to obtain images of an object for operation (e.g., organs). If an image of the object for operation cannot be obtained from the retractable camera 17a, the objective point extraction unit 62 cannot extract an objective point in an image from the retractable camera 17a and thus cannot confirm correspondency of the objective point between the image from the retractable camera 17a and an image from the retractable camera 17b and an image from the laparoscope 5. Further, the camera position estimation unit 64 cannot estimate a three-dimensional position of the camera 17a because it is provided that a positional relationship between the mark 19 and the retractable camera 17 is invariant. The depth estimation unit 65 cannot estimate a depth, and the image combining unit 66 cannot combine images.

As a result thereof, the three-dimensional image that is displayed on the three-dimensional monitor 7 may include noises. Because of unsuitable input of images and positional information, a burden of arithmetic processing of the image processing device 6 increases to an undesirable level.

If a three-dimensional image is created based on only images from the retractable camera 17b and the laparoscope 5, an amount of information decreases and precision falls.

In the present embodiment, the partial interruption instruction unit 71 is activated to change over an image to an image at the stored position, and the last image at the deployed position (several images including an image just before changed-over) and a three-dimensional position of the camera 17a are temporarily used. Accordingly, the image processing device 6 appropriately continues the arithmetic processing, and thus the three-dimensional image is continuously displayed on the three-dimensional monitor 7.

Meanwhile, at the time, since an image from the camera 17a cannot be obtained in real time, strictly speaking, it is not possible to obtain a three-dimensional real-time image. However, considering that images from the retractable camera 17b and the laparoscope 5 can be obtained in real time, that a large difference is hardly conceivable since the lens cleaning operation takes only one second, and that forceps is not operated during the lens cleaning operation, a fact that the image is a pseudo-real-time image does not invite a serious problem. The surgeon feels that the three-dimensional real-time image is continuing and thus can continue the surgical procedure without feeling uncomfortable.

(2) When the retractable camera 17a is changed over from the stored position to the deployed position again, a real-time image from the retractable camera 17a can be used again. However, if the image processing device 6 starts arithmetic processing from the beginning without going through the next image preparation unit 68, a burden of arithmetic processing of the image processing device 6 increases.

In the present embodiment, when the image matching determination unit 72 is activated and confirms that the images match each other, the restart instruction unit 73 is activated to cause the next image preparation unit 68 to be activated. This enables reduction of the burden of arithmetic processing of the image processing device 6 as well as securing of precision.

Meanwhile, considering that a large difference is hardly conceivable since the lens cleaning operation takes only one second and that the camera always returns to the same position (deployed position) by energization of the torsion spring 22, the images match each other in almost every cases, and thus the restart instruction unit 73 and the next image preparation unit 68 are activated.

(3) Meanwhile, the camera position estimation unit 64 estimates a camera position based on a mark position because it is provided that a positional relationship between the mark 19 and the retractable camera 17 is invariant. Therefore, position estimation accuracy may be deteriorated due to change-over of the camera.

In the present embodiment, since the camera always returns to the same position (deployed position) by energization of the torsion spring 22, position estimation accuracy can be ensured.

### ˜Modification of System˜

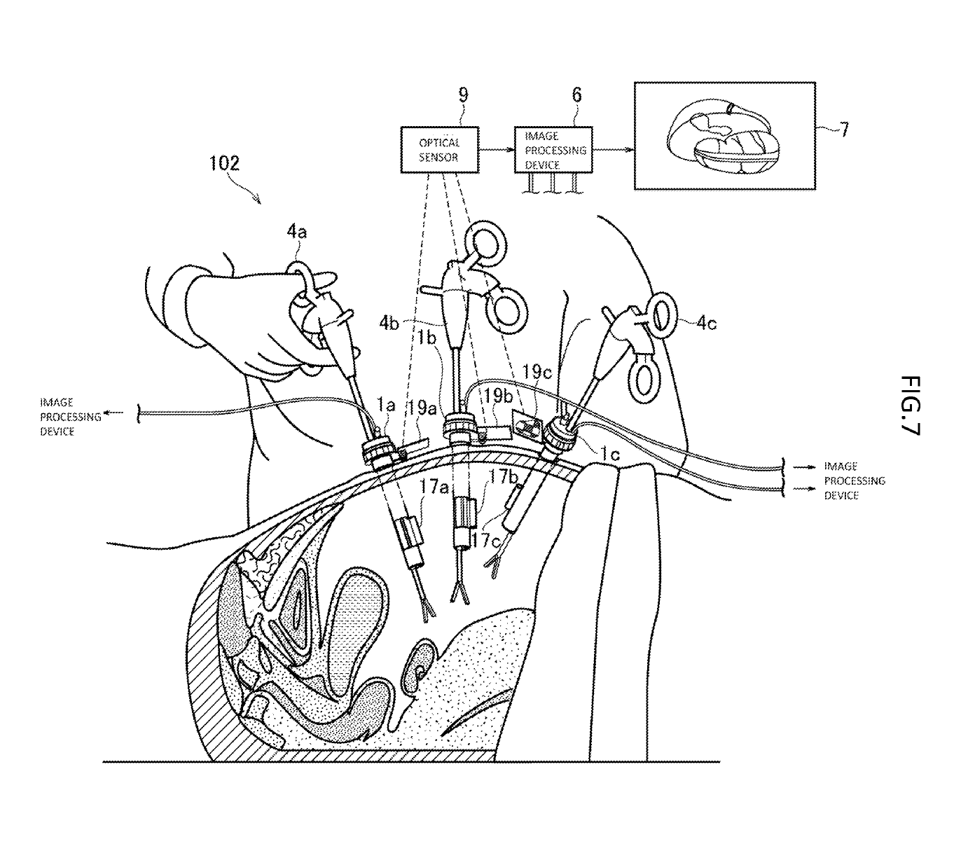

FIG. 7 is a schematic block diagram illustrating a surgery assistance system 102. The surgery assistance system 102 includes forceps trocars 1a, 1b, and 1c, forcipes 4a, 4b, and 4c, image processing device 6, and three-dimensional monitor 7. The forceps trocars 1a, 1b, and 1c include retractable cameras 17a, 17b, and 17c and marks 19a, 19b, and 19c, respectively. The image processing device 6 estimates a three-dimensional position of the respective cameras 17a, 17b, and 17c based on the three-dimensional positions of the marks 19a, 19b, and 19c, and creates a three-dimensional image by combining images obtained from the cameras. The three-dimensional monitor 7 outputs the three-dimensional image created by the image processing device 6.

More specifically, in the surgery assistance system 102, the trocars 3 for laparoscope, the laparoscope 5, and the mark 19d in the surgery assistance system 101 are omitted, and the forceps trocar 1c having the retractable camera 17c, the forceps 4c, and the mark 19c are newly added.

Meanwhile, in the laparoscopic surgery, a plurality of forcipes are normally used, however; it is possible that at least more than two forcipes and forceps trocars are included in the present system.

In a case where the laparoscope 5 is used as similar to the surgery assistance system 101, the surgeon needs to search a portion to be cut by operating an orientation of the laparoscope 5, whereas the retractable camera 17 can surely obtain an important image of, for example, a portion to be cut since the retractable camera 17 captures an image of a tip portion of the forceps 4a for sure. Therefore, it is possible to surely obtain an image of quality higher than that of the laparoscope 5, provided that the retractable camera 17 has high performance (preferably has performance at least almost equivalent to that of the laparoscope 5).

However, in order to unnecessitate the laparoscope 5, it is necessary to provide an alternative light source to the retractable camera 17.

Furthermore, since the trocar 3 for laparoscope and the laparoscope 5 are unnecessitated, it also becomes unnecessary to make a hole in an abdominal wall of a patient. This enhances low invasiveness.

Further, when the laparoscope 5 is unnecessitated, cleaning of the laparoscope 5 also becomes unnecessary. This can reduce a burden put upon a surgeon.

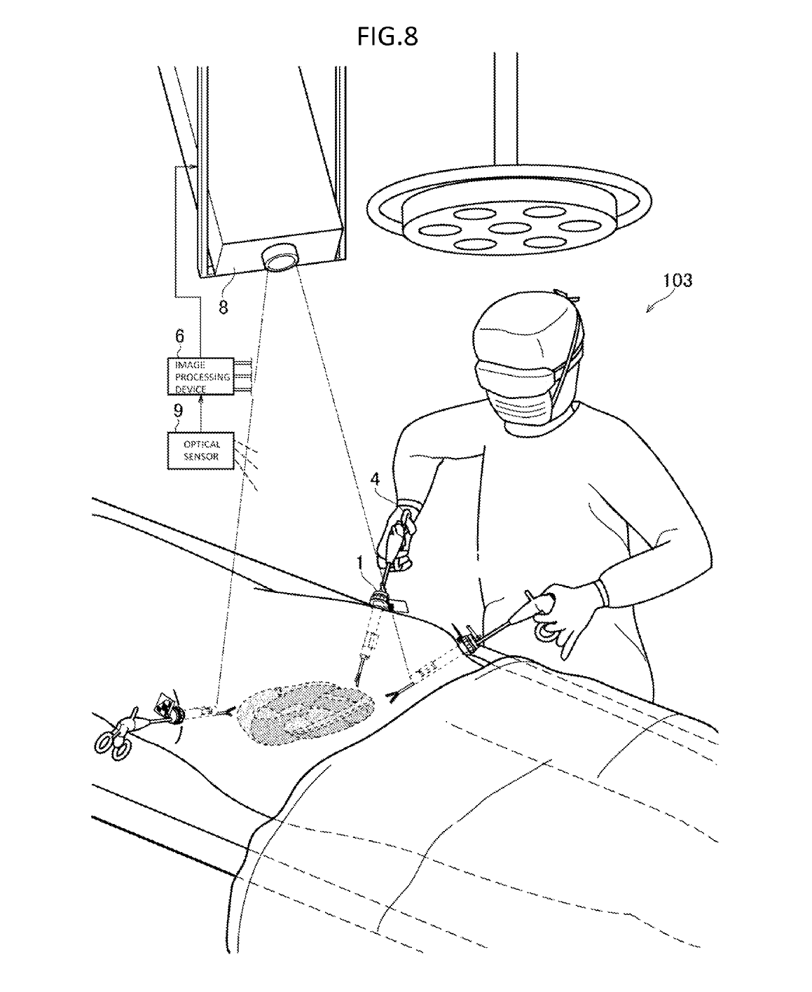

FIG. 8 is a schematic block diagram illustrating a surgery assistance system 103 as another modification. The surgery assistance system 103 is a modification of the surgery assistance systems 101 and 102. Elements common to those of the surgery assistance systems 101 and 102 are omitted for simplification.

In the surgery assistance systems 101 and 102, the surgeon performs surgery by operating the forcipes 4 and the laparoscope 5 while observing the monitor 7. However, this gives the surgeon a feeling of inconsistency of direction between eyes of the surgeon and an actual surgical field and an uncomfortable feeling, resulting in putting a burden upon the surgeon. Specifically, the surgeon having a good experiences in abdominal surgery may not be familiar to the laparoscopic surgery.

A surgery assistance system 103 includes a three-dimensional projector 8 instead of or together with the three-dimensional monitor 7. The three-dimensional projector 8 is provided above the operating table and directly projects a three-dimensional image created by the image processing device 6 upon an abdominal region of a patient.

Accordingly, the direction of the eyes of the surgeon matches the direction of the surgical field and reality similar to the abdominal surgery can be expressed by this. In other words, a burden put upon a surgeon can be decreased.

### <Port Including Retractable Camera>

Hereinabove, description is made provided that the laparoscopic surgery is performed. However, the present invention is also applicable to a thoracoscopic surgery. A surgical instrument referred to as trocar in laparoscopic surgery is called as port in thoracoscopic surgery. In other words, a trocar is almost the same medical instrument as a port.

### REFERENCE CHARACTER LIST

1 trocar
2 trocar (modification)
3 trocar (for laparoscope)
4 forceps
5 laparoscope
6 image processing device
7 three-dimensional monitor
8 three-dimensional projector
9 optical tracking sensor
11 pipe portion
12 head portion
13 opening portion
14 shaft
15 bearing
16 changing over mechanism
17 camera
18 cable
19 mark
21 wiper blade
22 torsion spring
25 changed-over position detection sensor
31 hinge mechanism
32 torsion spring
33 tension cable
61 image inputting unit
62 objective point extraction unit
63 mark position input unit
64 camera position estimation unit
65 depth estimation unit
66 image combining unit
67 image outputting unit
68 next image preparation unit
71 partial interruption instruction unit
72 image matching determination unit
73 restart instruction unit
101˜102 surgery assistance system

1. A trocar comprising:
a pipe portion that inserts a surgical instrument into an interior of a body of a patient;
a side opening portion that is provided at a position of said pipe portion that, during surgery, is within the body of the patient;
a retractable camera that is rotatably disposed so that it can be changed over between a stored position in which it is stored within the pipe portion and a deployed position in which it is deployed via said side opening portion to an exterior of said pipe portion and is capable of obtaining an image; and
a lens cleaning unit that contacts a camera lens for cleaning while said retractable camera is rotated.

2. The trocar according to claim 1:
wherein said lens cleaning unit includes a base portion and a tip portion, the base portion being fixed to a tip side of said side opening portion, the tip portion being deformable when the tip portion contacts said camera lens.

3. The trocar according to claim 1, further comprising:
an energizing unit that energizes said retractable camera toward the deployed position.

4. A surgery assistance system comprising:
a trocar according to claim 1;
a laparoscope; and
an image processing device that performs processing to combine an image obtained from said laparoscope and an image obtained from said retractable camera;
wherein said image processing device comprises
a partial interruption instruction unit that provides an instruction, when said retractable camera is changed from the deployed position to the stored position, to use an image lastly obtained at the deployed position.

5. A surgery assistance system comprising:
a plurality of trocars according to claim 1; and
an image processing device that performs processing to combine images obtained from said plurality of retractable cameras;
wherein said image processing device comprises
a partial interruption instruction unit that provides an instruction, when one of said plurality of retractable cameras is changed from the deployed position to the stored position, to use an image lastly obtained at the deployed position.

6. The surgery assistance system according to claim 4:
wherein said image processing device comprises
an image matching determination unit that determines whether or not the last image matches newly obtained latest image when said retractable camera is returned from the stored position to the deployed position; and
a restart instruction unit that provides an instruction to use the latest image when said image matching determination unit confirms matching of images.

7-15. (canceled)

16. The surgery assistance system according to claim 5:
wherein said image processing device comprises
an image matching determination unit that determines whether or not the last image matched newly obtained latest image when said retractable camera is returned from the stored position to the deployed position; and
a restart instruction unit that provides an instruction to use the latest image when said image matching determination unit confirms matching of images.

17. The surgery assistance system according to claim 4, further comprising:
a projector that is provided above an operating table, and that projects the combined image onto a position corresponding to an abdomen of the patient.

18. The surgery assistance system according to claim 5, further comprising:
a projector that is provided above an operating table, and that projects the combined image onto a position corresponding to an abdomen of the patient.

19. A port comprising:
a pipe portion that inserts a surgical instrument into a lung of a patient and being provided in a chest wall via said pipe portion;
a side opening portion that is provided in a position of said pipe portion that is within the lung of the patient;
a retractable camera that is rotatably disposed so that it can be changed over between a stored position in which it is stored within the pipe portion and a deployed position in which it is deployed via said side opening portion to the exterior of the pipe portion and is capable of obtaining and image; and
a cleaning unit that contacts a camera lens for cleaning while said retractable camera is rotated.

20. A trocar, comprising:
a pipe portion that inserts a surgical instrument into an interior of a body of a patient,
a retractable camera that is changeable between a stored position in which it is stored within said pipe portion and a deployed position in which it is deployed to an exterior of said pipe portion and is capable of obtaining an image; and
a camera cleaning unit that contacts a surface of said retractable camera for cleaning while the position of said retractable camera is changed.
